(19) United States
(12) Patent Application Publication
(10) Pub. No.: US 2026/0083919 A1
(43) Pub. Date: Mar. 26, 2026

(54) NEEDLE MODULE CAPABLE OF INJECTING MEDICINE

(71) Applicant: AGNES MEDICAL CO.,LTD, Seongnam-si (KR)

(72) Inventors: Gunyoung AHN, Seongnam-si (KR); Hyuck Ki HONG, Yongin-si (KR); Kyoung Su JUNG, Seongnam-si (KR); Moon ki SHIN, Seongnam-si (KR); Byoung Jun CHA, Namyangju-si (KR)

(73) Assignee: AGNES MEDICAL CO.,LTD, Seongnam-si (KR)

(21) Appl. No.: 19/085,103

(22) Filed: Mar. 20, 2025

(30) Foreign Application Priority Data
Sep. 20, 2024 (KR) 10-2024-0127709

(51) Int. Cl. A61M 5/32; A61M 5/34; A61N 1/32

(52) U.S. Cl. CPC A61M 5/3298; A61M 5/344; A61N 1/327; A61M 2205/0233

(57) ABSTRACT

A needle module includes a needle fixing unit including a first surface contacting the skin, a second surface opposite the first surface, needle seating holes, and medicine discharge holes, needles disposed in the needle seating holes and having tips, an energy transfer plate including a third surface in contact with ends of the needles opposite the tips, a fourth surface opposite the third surface, and through-holes formed corresponding to the medicine discharge holes, a medicine delivery channel unit including a fifth surface in contact with the fourth surface, a sixth surface opposite the fifth surface, medicine delivery holes formed corresponding to the through-holes, a medicine injection area, and a medicine delivery groove connecting the medicine injection area to the medicine delivery holes, and a Luer lock unit coupled to the needle fixing unit and including a medicine delivery passage to supply medicine to the medicine injection area.

### CROSS-REFERENCE TO PRIOR APPLICATION

This Application claims priority to Korean Patent Application No. 10-2024-0127709 (filed on September 20, 2024), which is hereby incorporated by reference in its entirety.

### BACKGROUND

The present disclosure relates to a needle module capable of injecting medicine used for skin treatment, and more particularly to a needle module including a needle configured to supply medicine to skin and also to be inserted into the skin so as to apply electrical energy to the skin, thereby enabling skin treatment using both medicine and high frequency.

In general, high-frequency current refers to alternating current of about 100 kHz or greater. High-frequency current has a short vibration width, and thus does not cause electrochemical reaction or an electrolysis phenomenon, and vibration current energy is converted into thermal energy only within a predetermined path.

In particular, when high-frequency current flows through the human body, because the vibration width of the high-frequency current is very short, ionic motion hardly occurs, and electrochemical reaction or an electrolysis phenomenon is not caused. When high-frequency electrical energy is applied to tissue, molecules constituting the tissue vibrate and rub against each other whenever the direction of the current changes, and thus generate bioheat.

In addition, unlike other types of currents, high-frequency current may generate thermal energy without stimulating sensory nerves and motor nerves and causing discomfort or muscle contraction in the human body, and this thermal energy may exhibit additional effects of enhancing functions of cells and increasing blood flow.

Accordingly, high-frequency treatment devices using high-frequency current have been used for various cosmetic treatment purposes such as skin regeneration, skin lifting, suppression of melanin pigment secretion, obesity treatment, skin care, hair promotion, and pain relief.

A general high-frequency cosmetic treatment device includes a handpiece having a high-frequency electrode, which is formed to come into contact with a treatment site of skin and to which a high-frequency current is applied, and a main body including a high-frequency generator configured to receive power and supply a high-frequency current to the handpiece and a high-frequency operation controller configured to control operation of the high-frequency generator. The handpiece is electrically connected to the main body through a cable or the like, and a needle formed to be inserted into skin of a treatment subject is provided at an end portion of the handpiece.

Such a general high-frequency cosmetic treatment device is being researched and developed for use with medicine in order to further enhance effects of relieving pain and improving the skin condition of a cosmetic treatment subject.

A conventional skin treatment device using both high frequency and medicine is configured such that medicine is separately applied to a treatment site or such that some of needles formed to be inserted into skin to apply high-frequency energy to the skin are implemented as needles having a cavity formed therein so as to inject medicine into the skin.

Such a conventional skin treatment device using both high frequency and medicine has a problem in that a separate treatment process of applying medicine to the skin should be added or in that it is impossible to reduce the length of the needles for injection of medicine to a certain length or less due to the shape and structural limitations of the needles.

### SUMMARY

An aspect of the present disclosure is directed to providing a needle module including a needle configured to supply medicine to a treatment site of skin and also to be inserted into the skin so as to apply electrical energy to the skin, thereby enabling effective skin treatment using both high frequency and medicine.

In accordance with the present disclosure, the above and other aspects can be accomplished by the provision of a needle module capable of injecting medicine, the needle module including a needle fixing unit including a first surface formed to be in contact with the skin during skin treatment and a second surface formed opposite the first surface and including a plurality of needle seating holes and a plurality of medicine discharge holes formed through the first surface and the second surface, a plurality of needles disposed in the plurality of needle seating holes, the plurality of needles having tips exposed outside the first surface, an energy transfer plate including a third surface formed to be in contact with ends of the plurality of needles opposite the tips, a fourth surface formed opposite the third surface, and a plurality of through-holes formed through the third surface and the fourth surface in an area corresponding to the plurality of medicine discharge holes, a medicine delivery channel unit including a fifth surface formed to be in contact with the fourth surface so as to cover the area in which the plurality of through-holes is formed, a sixth surface formed opposite the fifth surface, a plurality of medicine delivery holes formed through the fifth surface and the sixth surface in an area corresponding to the plurality of through-holes, a predetermined medicine injection area formed in the sixth surface, and a medicine delivery groove formed in the sixth surface so as to connect the medicine injection area to the plurality of medicine delivery holes, and a Luer lock unit coupled to the needle fixing unit so as to maintain the arrangement structure of the plurality of needles, the energy transfer plate, and the medicine delivery channel unit, the Luer lock unit including a medicine delivery passage formed therein so as to be connected to an external medicine supply device to receive medicine and to supply the medicine to the medicine injection area.

In the embodiment of the present disclosure, each of the plurality of needles may include a plate that is made of a conductive material and a plurality of prongs that is made of the same material as the plate, is integrally formed with the plate, and extends from one end of the plate in the same direction as each other. Tips formed at the plurality of prongs may be exposed outside the first surface.

In the embodiment of the present disclosure, the plurality of prongs may include a prong having a first length allowing a tip thereof to reach a papillary dermis of the skin when penetrating the skin and a prong having a second length allowing a tip thereof to reach a reticular dermis of the skin when penetrating the skin.

In the embodiment of the present disclosure, the energy transfer plate may include a recess or a hole formed therein so as to fix the ends of the plurality of needles opposite the tips, thereby fixing the positions of the plurality of needles.

In the embodiment of the present disclosure, the medicine delivery channel unit may be made of silicone.

In the embodiment of the present disclosure, the Luer lock unit may include a fastening portion protruding in a direction opposite the medicine delivery channel unit so as to be fastened to the external medicine supply device, and may supply medicine injected thereinto through a cavity formed in the fastening portion to the medicine injection area of the medicine delivery channel unit.

In the embodiment of the present disclosure, the Luer lock unit may include a sidewall completely surrounding a side surface of the medicine delivery channel unit and a plurality of fixing shafts protruding toward the needle fixing unit. The plurality of fixing shafts may be tightly fitted into fixing recesses formed in the needle fixing unit so that the Luer lock unit and the needle fixing unit are coupled to each other.

In the embodiment of the present disclosure, in a coupled state of the Luer lock unit to the needle fixing unit, an edge area of the fourth surface of the energy transfer plate may be exposed in the lateral direction of the Luer lock unit.

The embodiment of the present disclosure may further include a terminal unit formed so as to allow the fastening portion of the Luer lock unit to pass therethrough and to surround the Luer lock unit while being in contact with the fourth surface exposed in the lateral direction of the Luer lock unit, and the terminal unit may have a surface made of an electrically conductive material.

The embodiment of the present disclosure may further include a cover unit disposed so as to surround a lateral side of the terminal unit to prevent leakage of high-frequency electrical energy transferred to the terminal unit.

### BRIEF DESCRIPTION OF THE DRAWINGS

The above and other aspects, features, and other advantages of the present disclosure will be more clearly understood from the following detailed description taken in conjunction with the accompanying drawings, in which:

FIG. 1 is a perspective view of a needle module capable of injecting medicine according to an embodiment of the present disclosure;

FIG. 2 is an exploded perspective view of the needle module capable of injecting medicine according to the embodiment of the present disclosure;

FIG. 3 is a front view of the needle module capable of injecting medicine according to the embodiment of the present disclosure;

FIG. 4 is a cut-away perspective view showing a cross-section cut along a medicine injection hole in the needle module capable of injecting medicine according to the embodiment of the present disclosure; and

FIG. 5 is a cut-away perspective view showing a plate-shaped needle of the needle module capable of injecting medicine according to the embodiment of the present disclosure.

### DETAILED DESCRIPTION

Hereinafter, embodiments of the present disclosure will be described in detail with reference to the accompanying drawings so that those skilled in the art to which the present disclosure pertains may easily carry out the embodiments. The present disclosure may, however, be embodied in many different forms, and should not be construed as being limited to the embodiments set forth herein. In the drawings, illustration of parts unrelated to the description is omitted to clearly and briefly describe the present disclosure, and the same or extremely similar components are denoted by the same reference numerals throughout the specification.

It is to be understood that the phraseology and terminology used in the following specification and appended claims should not be construed as being limited to general and dictionary meanings but be construed as the meanings and concepts according to the spirit of the present disclosure on the basis of the principle that the inventor is permitted to define appropriate terms and concepts for the best explanation.

Therefore, embodiments described in the specification and the configuration of elements illustrated in the drawings are merely considered to be preferred embodiments and do not represent all the technical ideas of the present disclosure, and thus it should be understood that various equivalents and modifications may exist at the time of filing this application.

It will be further understood that the term “include” or “have”, when used herein, specifies the presence of stated features, integers, steps, operations, elements, components, or combinations thereof, but does not preclude the presence or addition of one or more other features, integers, steps, operations, elements, components, or combinations thereof.

When an element is referred to as being disposed “in front of,” “behind,” “above,” or “below” another element, it includes not only a case in which the element is disposed “in front of,” “behind,” “above,” or “below” the other element so as to be in direct contact therewith but also a case in which intervening elements are present, unless stated otherwise. In addition, when an element is referred to as being “connected” or “coupled” to another element, it includes not only a case in which the element is directly “connected” or “coupled” to the other element but also a case in which the element is indirectly “connected” or “coupled” to the other element, unless stated otherwise.

Hereinafter, a needle module capable of injecting medicine according to an embodiment of the present disclosure will be described with reference to the accompanying drawings.

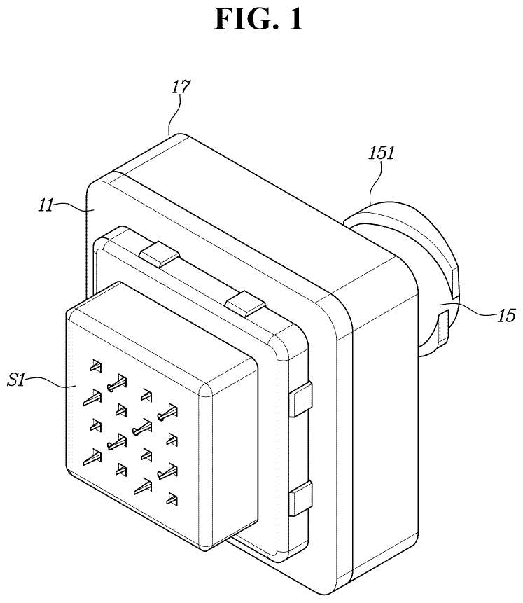

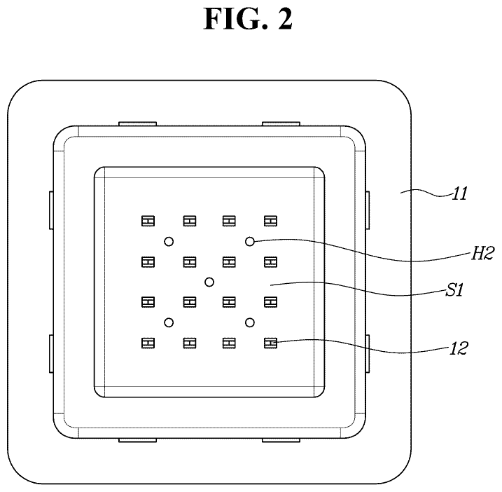

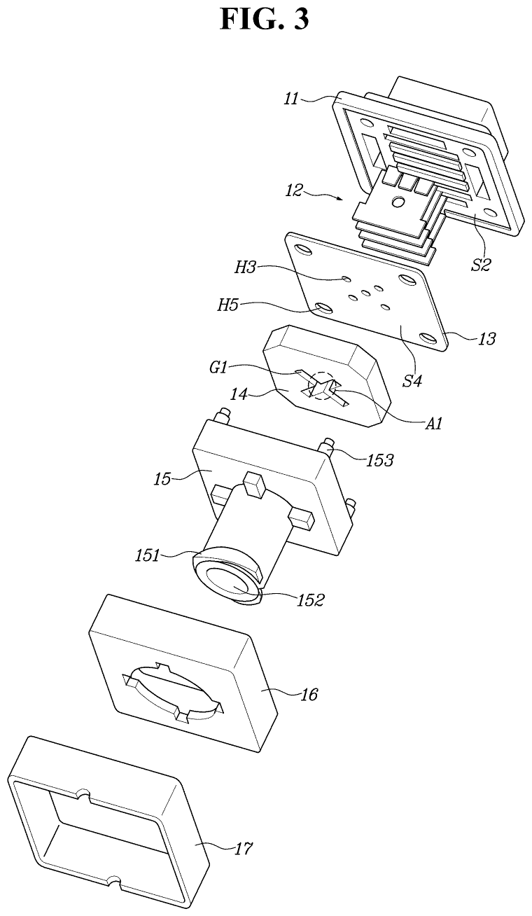

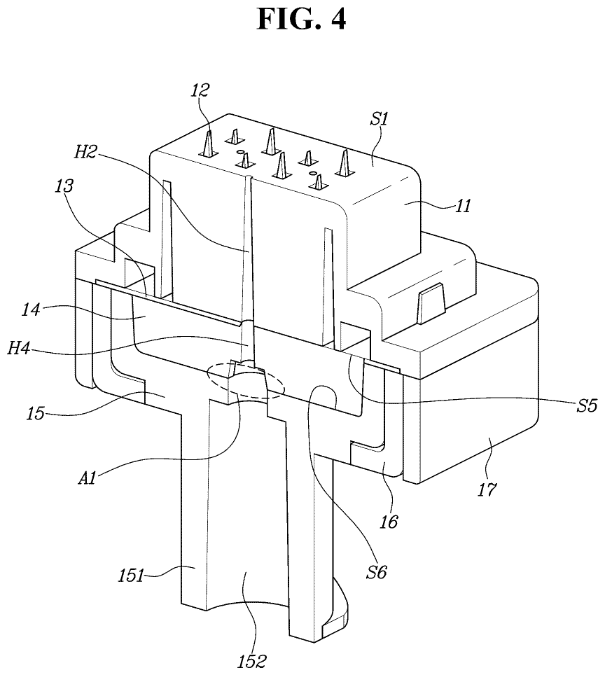

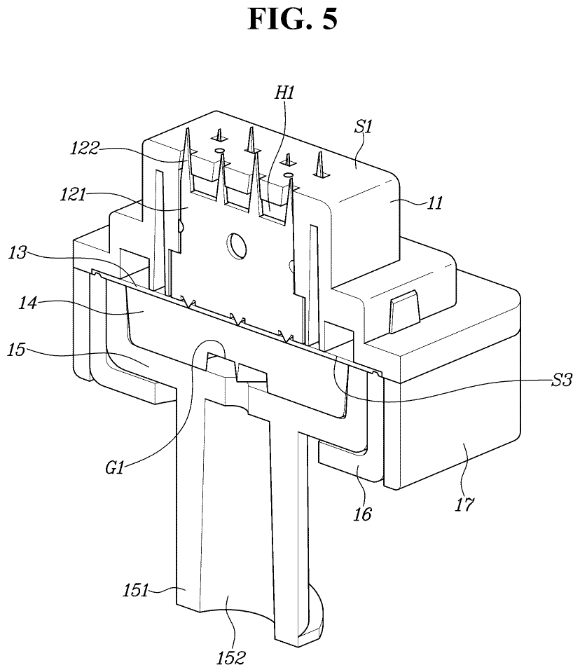

FIG. 1 is a perspective view of a needle module capable of injecting medicine according to an embodiment of the present disclosure. FIG. 2 is an exploded perspective view of the needle module capable of injecting medicine according to the embodiment of the present disclosure. FIG. 3 is a front view of the needle module capable of injecting medicine according to the embodiment of the present disclosure. FIG. 4 is a cut-away perspective view showing a cross-section cut along a medicine injection hole in the needle module capable of injecting medicine according to the embodiment of the present disclosure. FIG. 5 is a cut-away perspective view showing a plate-shaped needle of the needle module capable of injecting medicine according to the embodiment of the present disclosure.

Referring to FIGS. 1 to 5, the needle module capable of injecting medicine according to an embodiment of the present disclosure may include a needle fixing unit 11, a needle 12, an energy transfer plate 13, a medicine delivery channel unit 14, and a Luer lock unit 15.

In addition, the needle module capable of injecting medicine according to the embodiment of the present disclosure may further include a terminal unit 16 and a cover unit 17.

The needle fixing unit 11 may include a first surface S1 formed to be in contact with the skin during skin treatment and a second surface S2 formed opposite the first surface S1, and may include a plurality of needle seating holes H1 and a plurality of medicine discharge holes H2 formed through the first and second surfaces S1 and S2.

The first surface S1 may be a skin contact surface that comes into direct contact with the skin, which is a target to be treated. During the skin treatment, the medicine discharged from the medicine discharge holes H2 may be coated on a tip of the needle 12 exposed outside each of the needle seating holes H1, and then may penetrate the skin together with the tip of the needle 12.

The plurality of medicine discharge holes H2 may be formed in the needle fixing unit 11 so as to extend straight between the first surface S1 and the second surface S2 of the needle fixing unit 11, and the plurality of needle seating holes H1 may be formed in the needle fixing unit 11 so as to have a structure corresponding to the shape of the needle 12.

The needle 12 may be disposed in each of the plurality of needle seating holes H1, and the tip of the needle 12 may be exposed outside the first surface S1 of the needle fixing unit 11. The needle 12 may be implemented as an individual needle having a conical or cylindrical shape. However, in the embodiment of the present disclosure, the needle 12 may be a plate-shaped needle, which includes a plate 121 and a plurality of prongs 122 extending from the plate 121 to form respective tips.

In more detail, the plate-shaped needle 12 applied to the embodiment of the present disclosure may include a plate 121, which is made of a conductive material, and a plurality of prongs 122, which is made of the same material as the plate 121, is integrally formed with the plate 121, and extends in the same direction as each other from one end of the plate 121.

The plate 121 may be made of a conductive material through which a current is capable of flowing, e.g., a single metallic material or an alloy of a plurality of metals. The shape of the plate 121 may be changed to various shapes as needed under various conditions such as an environment in which the plate 121 is employed or the shapes of neighboring structures.

The plate 121 may have an area to which an electrical signal is applied. The area to which an electrical signal is applied may be variously changed depending on a form in which the plate-shaped needle 12 is assembled. In the embodiment of the present disclosure, a plurality of plate-shaped needles 12 may be assembled so as to be disposed parallel to each other, whereby an end portion of the plate 121 opposite the end portion at which the prongs 122 are formed may be an area to which an electrical signal is applied. That is, the opposite end portion of the plate 121 of the plate-shaped needle 12 may be in contact with the energy transfer plate 13 to be described later, and accordingly, an electrical signal may be applied to the plate 121 of the plate-shaped needle 12 from the energy transfer plate 12.

The prongs 122 extending from the plate 121 may be formed in a pin or blade shape. The prongs 122 may be formed of the same material as the plate 121 and may be integrally formed with the plate 121. That is, during the manufacturing process, the prongs 122 and the plate 121 may be manufactured simultaneously through a single process.

The prongs 122 may be exposed outside the first surface S1 of the needle fixing unit 11 so as to be inserted into the skin. The prongs 122 may extend from the plate 121 by the same length or different lengths. In the embodiment shown in the drawings, the prongs 122 may extend from the plate 121 by two different lengths. In detail, the prong having a relatively short length may be formed so that the tip thereof reaches the papillary dermis layer, and the prong having a relatively long length may be formed so that the tip thereof reaches the reticular dermis layer.

The energy transfer plate 13 may be in contact with the end portion of the needle 12 opposite the tips of the needle 12 to transfer an electrical signal (electrical energy) to the needle 12. In more detail, the energy transfer plate 13 may include a third surface S3 formed to be in contact with the end portion of the needle 12 opposite the tips of the needle 12, a fourth surface S4 formed opposite the third surface S3, and a plurality of through-holes H3 formed through the third and fourth surfaces S3 and S4.

The energy transfer plate 13 may be made of an electrically conductive metallic material. In detail, although not shown in the drawings, the energy transfer plate 13 may include a recess or a hole formed therein so as to fix the end portion of the needle 12 opposite the tips of the needle 12, thereby fixing the position of the needle 12.

The energy transfer plate 13 may serve as a terminal to transfer electrical energy (an electrical signal) supplied from the outside to the needle 12.

In addition, the energy transfer plate 13 may include a plurality of through-holes H3 formed in an area corresponding to the medicine discharge holes H2 so as to deliver the medicine supplied from the outside to the medicine discharge holes H2.

The medicine delivery channel unit 14 may include a fifth surface S5 formed to be in contact with the fourth surface S4 of the energy transfer plate 13 so as to cover the area in which the plurality of through-holes H3 is formed, a sixth surface S6 formed opposite the fifth surface S5, and a plurality of medicine delivery holes H4 formed through the fifth and sixth surfaces S5 and S6 in an area corresponding to the plurality of through-holes H3. A predetermined medicine injection area A1 and a medicine delivery groove G1 connecting the medicine injection area A1 to the plurality of medicine delivery holes H4 may be formed in the sixth surface S6.

The medicine delivery channel unit 14 may be formed of a material having excellent contact properties and constant elasticity, such as silicone. The medicine delivery channel unit 14 may include a fifth surface S5 that is in contact with the fourth surface S4 of the energy transfer plate 13, a sixth surface S6 that is a surface opposite the fifth surface S5, and medicine delivery holes H4 formed in an area corresponding to the through-holes H3 in the energy transfer plate 13.

In addition, a medicine injection area A1, to which medicine is supplied from the outside, may be formed in the sixth surface S6 of the medicine delivery channel unit 14. In addition, a medicine delivery groove G1 may be formed in the sixth surface S6 of the medicine delivery channel unit 14 so as to be located between the medicine injection area A1 and the medicine delivery holes H4. Accordingly, if medicine is supplied to the medicine injection area A1 from the outside, the medicine may be distributed to the medicine delivery holes H4 along the medicine delivery groove G1. The distributed medicine may be discharged outside the first surface S1 of the needle fixing unit 11 through the medicine delivery holes H4, the through-holes H3, and the medicine discharge holes H2.

The Luer lock unit 15 may be coupled to the needle fixing unit 11 so as to maintain the arrangement structure of the needle 12, the energy transfer plate 13, and the medicine delivery channel unit 14 described above, and may include a medicine delivery passage formed therein so as to be connected to an external medicine supply device (e.g., syringe) to receive medicine and to supply the medicine to the medicine injection area A1 of the medicine delivery channel unit 14.

The Luer lock unit 15 may include a fastening portion 151 protruding in a direction opposite the medicine delivery channel unit 14 so as to be fastened to a syringe (not shown). The Luer lock unit 15 may supply the medicine injected thereinto through a cavity 152 formed in the fastening portion 151 to the medicine injection area A1 formed in the sixth surface S6 of the medicine delivery channel unit 14.

The Luer lock unit 15 may include a sidewall completely surrounding the side surface of the medicine delivery channel unit 14 and a plurality of fixing shafts 153 protruding toward the needle fixing unit 11. The fixing shafts 153 may be tightly fitted into fixing recesses formed in the needle fixing unit 11, whereby the Luer lock unit 15 and the needle fixing unit 11 may be securely coupled to each other.

In addition, in the embodiment of the present disclosure, the energy transfer plate 13 may further include through-holes H5 formed therein so as to allow the fixing shafts 153 of the Luer lock unit 15 to pass therethrough. In the state in which the Luer lock unit 15 and the needle fixing unit 11 are coupled to each other, the edge area of the fourth surface S4 of the energy transfer plate 13 may be exposed in the lateral direction of the Luer lock unit 15.

The terminal unit 16 may be formed so as to allow the fastening portion 151 of the Luer lock unit 15 to pass therethrough and to surround the Luer lock unit 15 while being in contact with the fourth surface S4 of the energy transfer plate 13 exposed in the lateral direction of the Luer lock unit 15. The terminal unit 16 may be a structure made of an electrically conductive metallic material or a structure having a surface plated with an electrically conductive metal. The terminal unit 16 may be exposed in a direction opposite the Luer lock unit 15 and thus may function as a terminal that receives high-frequency electrical energy from an electrical signal generator provided in a handpiece or a main body of a treatment device.

The cover unit 17 may be disposed so as to surround the lateral side of the terminal unit 16, thereby preventing leakage of the high-frequency electrical energy transferred to the terminal unit 16.

As is apparent from the above description, unlike the conventional skin treatment device in which medicine is separately applied to the skin or is injected into the skin through a needle, the needle module capable of injecting medicine according to the embodiment of the present disclosure is configured such that a tip of a needle formed to penetrate the skin and a medicine discharge hole through which medicine is discharged outside are disposed together on a surface that is in contact with the skin for skin treatment, and accordingly, the medicine may be coated on the tip of the needle and may be injected into the skin simultaneously with insertion of the needle into the skin during skin treatment.

As a result, the needle module capable of injecting medicine according to the embodiment of the present disclosure may reduce personnel for skin treatment and may simplify the skin treatment process. Furthermore, the needle module capable of injecting medicine according to the embodiment of the present disclosure may overcome the structural limitations of a needle for medicine injection, thereby enabling use of needles having various lengths and thus exhibiting further improved skin treatment effects.

Although specific embodiments of the present disclosure have been disclosed for illustrative purposes, those skilled in the art will appreciate that various modifications, additions, and substitutions are possible, without departing from the scope and spirit of the disclosure as disclosed in the accompanying claims.

1. A needle module capable of injecting medicine, the needle module comprising:
a needle fixing unit comprising a first surface formed to be in contact with a skin during skin treatment and a second surface formed opposite the first surface and comprising a plurality of needle seating holes and a plurality of medicine discharge holes formed through the first surface and the second surface;
a plurality of needles disposed in the plurality of needle seating holes, the plurality of needles having tips exposed outside the first surface;
an energy transfer plate comprising a third surface formed to be in contact with ends of the plurality of needles opposite the tips, a fourth surface formed opposite the third surface, and a plurality of through-holes formed through the third surface and the fourth surface in an area corresponding to the plurality of medicine discharge holes;
a medicine delivery channel unit comprising a fifth surface formed to be in contact with the fourth surface so as to cover the area in which the plurality of through-holes is formed, a sixth surface formed opposite the fifth surface, a plurality of medicine delivery holes formed through the fifth surface and the sixth surface in an area corresponding to the plurality of through-holes, a predetermined medicine injection area formed in the sixth surface, and a medicine delivery groove formed in the sixth surface so as to connect the medicine injection area to the plurality of medicine delivery holes; and
a Luer lock unit coupled to the needle fixing unit so as to maintain an arrangement structure of the plurality of needles, the energy transfer plate, and the medicine delivery channel unit, the Luer lock unit comprising a medicine delivery passage formed therein so as to be connected to an external medicine supply device to receive medicine and to supply the medicine to the medicine injection area.

2. The needle module according to claim 1, wherein each of the plurality of needles comprises:
a plate made of a conductive material; and
a plurality of prongs made of a same material as the plate, the plurality of prongs being integrally formed with the plate, the plurality of prongs extending from one end of the plate in a same direction as each other, and
wherein tips formed at the plurality of prongs are exposed outside the first surface.

3. The needle module according to claim 2, wherein the plurality of prongs comprises:
a prong having a first length allowing a tip thereof to reach a papillary dermis of the skin when penetrating the skin; and
a prong having a second length allowing a tip thereof to reach a reticular dermis of the skin when penetrating the skin.

4. The needle module according to claim 1, wherein the energy transfer plate comprises a recess or a hole formed therein so as to fix the ends of the plurality of needles opposite the tips, thereby fixing positions of the plurality of needles.

5. The needle module according to claim 1, wherein the medicine delivery channel unit is made of silicone.

6. The needle module according to claim 1, wherein the Luer lock unit comprises a fastening portion protruding in a direction opposite the medicine delivery channel unit so as to be fastened to the external medicine supply device, and supplies medicine injected thereinto through a cavity formed in the fastening portion to the medicine injection area of the medicine delivery channel unit.

7. The needle module according to claim 1, wherein the Luer lock unit comprises:
a sidewall completely surrounding a side surface of the medicine delivery channel unit; and
a plurality of fixing shafts protruding toward the needle fixing unit, and
wherein the plurality of fixing shafts is tightly fitted into fixing recesses formed in the needle fixing unit so that the Luer lock unit and the needle fixing unit are coupled to each other.

8. The needle module according to claim 1, wherein, in a coupled state of the Luer lock unit to the needle fixing unit, an edge area of the fourth surface of the energy transfer plate is exposed in a lateral direction of the Luer lock unit.

9. The needle module according to claim 8, further comprising a terminal unit formed so as to allow the fastening portion of the Luer lock unit to pass therethrough and to surround the Luer lock unit while being in contact with the fourth surface exposed in the lateral direction of the Luer lock unit, the terminal unit having a surface made of an electrically conductive material.

10. The needle module according to claim 9, further comprising a cover unit disposed so as to surround a lateral side of the terminal unit to prevent leakage of high-frequency electrical energy transferred to the terminal unit.
